(19) United States
(12) Patent Application Publication
(10) Pub. No.: US 2004/0120914 A1
(43) Pub. Date: Jun. 24, 2004

(54) Composition containing a quaternary silicone, a cation and two cationic polymers and method of use

(75) Inventors: Sandrine Decoster, Saint Gratien (FR); Benedicte Cazin, Clichy (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 10/608,264

(22) Filed: Jun. 30, 2003

Related U.S. Application Data
(60) Provisional application No. 60/393,832, filed on Jul. 8, 2002.

(30) Foreign Application Priority Data
Jun. 28, 2002 (FR) 0208142

(51) Int. Cl. A61K 7/06; A61K 7/11

(52) U.S. Cl. 424/70.12

(57) ABSTRACT

The present invention relates to a nonwashing cosmetic composition containing, in a cosmetically acceptable medium, at least one silicone containing quaternary ammonium groups, at least one cationic surfactant, at least two cationic polymers that are different from each other and at least one nonionic and nonassociative thickening polymer, and to a cosmetic process for treating keratin materials, in particular the hair. These compositions are transparent and have a fondant texture. Hair treated with these compositions is shiny, feels soft and is residue-free.

### REFERENCE TO PRIOR APPLICATIONS

This application claims priority to U.S. provisional application 60/393,832, filed Jul. 8, 2002, and to French patent application 0208142, filed Jun. 28, 2002, both of which are incorporated herein by reference.

### SUMMARY OF THE INVENTION

The present invention relates to a nonwashing composition, especially a hair conditioner, comprising a quaternary silicone, a cation, at least one nonionic thickening polymer and at least two cationic polymers, and to a process for treating keratin materials, in particular the hair. In a preferred embodiment the present invention relates to a nonwashing composition comprising, preferably in a cosmetically acceptable medium, at least one silicone containing quaternary ammonium groups, at least one cationic surfactant, at least two cationic polymers that are different than each other and at least one nonionic and nonassociative thickening polymer, and to a process for treating keratin materials with this composition, in particular the hair. Preferably these compositions are transparent and have a fondant texture. Hair treated with these compositions is shiny, feels soft and is residue-free.

### BACKGROUND OF THE INVENTION

It is well known that hair that has been sensitized (i.e. damaged and/or embrittled) to varying degrees under the action of atmospheric agents or under the action of mechanical or chemical treatments, such as dyeing, bleaching and/or permanent-waving, is often difficult to disentangle and to style, and lacks softness.

Cosmetic compositions containing thickening polysaccharides in particular such as starch or celluloses have already been proposed for treating keratin materials, and in particular the hair.

However, such compositions have drawbacks such as rinseability problems, stability problems, difficulties in distributing them over the keratin materials and also insufficient cosmetic properties.

It has already been recommended to use cationic polymers, cationic silicones or cationic surfactants in compositions for washing or caring for keratin materials such as the hair, to facilitate the disentangling of the hair and to give it softness and suppleness. The use of cationic polymers or cations for this purpose has various drawbacks. On account of their high affinity for the hair, some of these polymers become deposited in substantial amounts during repeated use, and lead to undesirable effects such as an unpleasant, laden feel, stiffening of the hair, and adhesion between the fibers that affects styling.

In summary, it is found that the current conditioning cosmetic compositions are not entirely satisfactory.

Moreover, it is occasionally sought to obtain transparent cosmetic compositions, which are particularly appreciated by consumers. The standard conditioning compositions based on fatty alcohols of the prior art are not transparent. The inventors have now discovered that the combination of a quaternary silicone, a cationic surfactant, at least two cationic polymers and a nonionic thickening polymer, especially in nondetergent media with a low or zero concentration of washing surfactants, makes it possible to overcome these drawbacks.

Hair treated with this composition is smooth, disentangles easily, is shiny, supple, individualized and has a soft, residue-free feel. The hair has a natural and unladen appearance. In addition, these compositions can be transparent and have a fondant texture, i.e. they disappear quickly into the hair.

### DETAILED DESCRIPTION OF THE PREFERRED EMBODIMENTS

Thus, according to the present invention, novel nonwashing compositions are provided, comprising, preferably in a cosmetically acceptable medium, at least one silicone containing quaternary ammonium groups, at least one cationic surfactant, at least two cationic polymers that are different than (from) each other, and at least one nonionic and nonassociative polymeric thickener.

Another subject of the invention is a process for treating keratin materials and in particular the hair, using the invention compositions.

Preferably, the compostions, methods and medium of the invention are cosmetic, or cosmetically acceptable, as the case may be. This is sometimes denoted herein by placing these terms in parentheses in order to make clear that these modifiers are preferred but not required.

A subject of the invention is also the use of the invention compositions as a conditioner.

A subject of the invention is also the use of the invention compositions to give the hair sheen.

A subject of the invention is also the use of the invention compositions to give the hair suppleness.

Other subjects, characteristics, aspects and advantages of the invention will become even more apparent on reading the description and the various examples that follow.

In accordance with the invention, the expression “silicone containing quaternary ammonium groups” means any silicone comprising one or more quaternary ammonium groups. These quaternary ammonium groups may be linked in the alpha or omega position in the form of side groups. They may be linked directly to the polysiloxane skeleton or may be borne by hydrocarbon-based chains.

According to the invention, in accordance with what is generally accepted, the term “silicone” means any polymer having a structure based on an alternation of silicon and oxygen atoms, linked together via bonds known as siloxane bonds (—Si—O—Si—), and also characterized by the existence of silicon-carbon bonds. These silicones, or polysiloxanes, are generally obtained by polycondensation of suitably functionalized silanes. The hydrocarbon-based radicals most commonly borne by the silicon atoms are lower alkyl radicals, in particular methyl, fluoroalkyl radicals and aryl radicals, in particular phenyl.

The silicones containing quaternary ammonium groups of the present invention include those corresponding to the following general formulae:

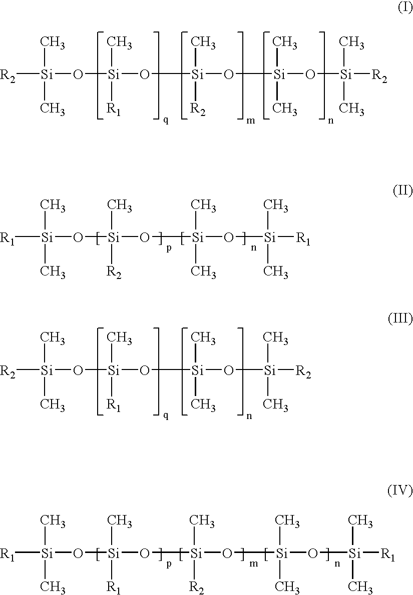

in which formulae:

R₁, which may be identical or different, represents a C₁-C₃₀ linear or branched alkyl group, or phenyl;

R₂, which may be identical or different, represents —C_{c}H_{2c}—O—(C₂H₄O)ₐ—(C₃H₆O)_{b}—R₅ or —C_{c}H_{2c}—O—(C₄H₈O)ₐ—R₅;

R₃ and R₄, which may be identical or different, each denote a C₁-C₁₂ linear or branched alkyl group, and preferably a methyl group;

R₅, which may be identical or different, is chosen from the groups having the following formula:

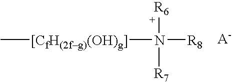

the radicals R₈ independently represent a linear or branched C₁₋₂₂ alkyl or C₂₋₂₂ alkenyl radical optionally bearing one or more OH groups, or represent a group CₕH₂ₕZCOR₉;

R₆, R₇ and R₉, which may be identical or different, represent linear or branched

C₁₋₂₂ alkyl or C₂₋₂₂ alkenyl radicals optionally bearing one or more OH groups, or

R₇ may form with a portion of R₈ a heterocycle (ring with at least one hetero atom, for instance N, O or P), the heterocycle is especially an imidazoline. Preferably, R₆ and R₇ denote a C₁-C₆ alkyl radical and more particularly methyl, R₉ preferably denotes a radical chosen from C₈-C₁₈ alkyl and C₈-C₁₈ alkenyl and especially a cocoyl radical,

m ranges from 0 to 20;

n ranges from 0 to 500;

p ranges from 1 to 50;

q ranges from 0 to 20;

a ranges from 0 to 50;

b ranges from 0 to 50;

c ranges from 0 to 4;

f ranges from 0 to 4;

g ranges from 0 to 2 and is preferably equal to 1;

h ranges from 1 to 4 and is preferably equal to 3;

Z represents an oxygen atom or NH,

A⁻ represents a monovalent mineral or organic anion such as a halide (chloride or bromide), a sulfate or a carboxylate (acetate, lactate or citrate).

The silicones containing a quaternary ammonium that are preferably used are those corresponding to the general formula (III) as defined above, and more particularly those corresponding to the general formula (III) in which at least one, and preferably all, of the following conditions are met:

c is equal to2 or3;

R₁ denotes a methyl group;

a and b are equal to zero;

n ranges from 0 to 100;

q is equal to 0;

f=3;

g=1;

R₆ and R₇ denote a methyl group;

R₈ denotes a radical —(CH₂)—NHCOR₉.

Such silicones are sold, for example, by the company Goldschmidt under the names Abil Quat 3272, Abil B 9905, Abil Quat 3474 and Abil K 3270, by the company Lipo France, under the names Silquat Q-100, Silquat Q-200 WS, Silquat AX, Silquat AC, Silquat AD and Silquat AM all manufactured by the company Siltech, by the company OSI under the name Magnasoft Exhaust and Silsoft C-880 and by the company UCIB under the names Pecosil 14-PQ and Pecosil 36-PQ (manufactured by Phoenix Chemical).

These silicones are described especially in patents EP 530 974, DE 3 719 086, DE 3 705 121, EP 617 607 and EP 714 654.

The silicones containing quaternary ammonium groups used in accordance with the invention can be in the form of aqueous solutions, in the form of dispersions or emulsions in water, etc.

In the compositions of the present invention, the silicone(s) containing quaternary ammonium groups is(are) preferably present in a proportion of from 0.01% to 10% by weight, more preferably in a proportion of from 0.1% to 5% by weight of active material, and more particularly from 0.1% to 1% by weight, relative to the total weight of the composition.

The composition according to the invention comprises one or more cationic surfactants including those that are well known per se, such as optionally polyoxyalkylenated primary, secondary or tertiary fatty amine salts, and mixtures thereof.

Useful quaternary ammonium salts that may especially be mentioned include:

those of general formula (V) below:

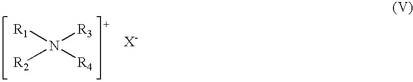

in which the symbols R₁ to R₄, which may be identical or different, represent a linear or branched aliphatic radical containing from 1 to 30 carbon atoms, or an aromatic radical such as aryl or alkylaryl. The aliphatic radicals may comprise hetero atoms such as, especially, oxygen, nitrogen, sulfur and halogens. The aliphatic radicals are chosen, for example, from alkyl, alkoxy, C₂-C₆ polyoxyalkylene, alkylamide, (C₁₂-C₂₂)alkylamido(C₂-C₆)alkyl, (C₁₂-C₂₂)alkyl-acetate and hydroxyalkyl radicals, containing from about 1 to 30 carbon atoms; X⁻ is an anion chosen from the group of halides, phosphates, acetates, lactates, (C₂-C₆ alkyl sulfates and alkyl- or alkylaryl-sulfonates;

quaternary ammonium salts of imidazoline, for instance those of formula (VI) below:

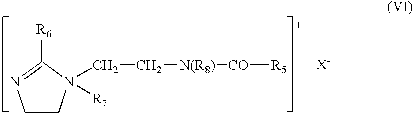

in which R₅ represents an alkenyl or alkyl radical containing from 8 to 30 carbon atoms, for example fatty acid derivatives of tallow or of coconut, R₆ represents a hydrogen atom, a C₁-C₄ alkyl radical or an alkenyl or alkyl radical containing from 8 to 30 carbon atoms, R₇ represents a C₁-C₄ alkyl radical, R₈ represents a hydrogen atom or a C₁-C₄ alkyl radical, and X is an anion chosen from the group of halides, phosphates, acetates, lactates, alkyl sulfates, alkyl sulfonates or alkylaryl sulfonates. R₅ and R₆ preferably denote a mixture of alkenyl or alkyl radicals containing from 12 to 21 carbon atoms, such as, for example, fatty acid derivatives of tallow, R₇ denotes methyl and R₈ denotes hydrogen. Such a product is, for example, Quaternium-27 (CTFA 1997) or Quaternium-83 (CTFA 1997), which are sold under the names “Rewoquat®” W75, W90, W75PG and W75HPG by the company Witco,

diquaternary ammonium salts of formula (VII):

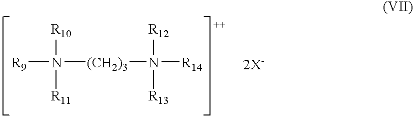

in which R₉ denotes an aliphatic radical containing from about 16 to 30 carbon atoms, R₁₀, R₁₁, R₁₂, R₁₃ and R₁₄, which may be identical or different, are chosen from hydrogen and an alkyl radical containing from 1 to 4 carbon atoms, and X- is an anion chosen from the group of halides, acetates, phosphates, nitrates and methyl sulfates. Such diquaternary ammonium salts in particular comprise propanetallowdiammonium dichloride;

quaternary ammonium salts containing at least one ester function, such as those of formula (VIII) below:

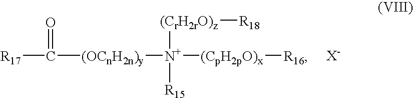

in which:

R₁₅ is chosen from C₁-C₆ alkyl radicals and C₁-C₆ hydroxyalkyl or dihydroxyalkyl radicals;

R₁₆ is chosen from:

a radical

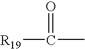

linear or branched, saturated or unsaturated C₁-C₂₂ hydrocarbon-based radicals R₂₀,

a hydrogen atom,

R₁₇ is chosen from:

a radical

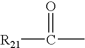

linear or branched, saturated or unsaturated C₁-C₆ hydrocarbon-based radicals R₂₂,

a hydrogen atom,

R₁₇, R₁₉ and R₂₁, which may be identical or different, are chosen from linear or branched, saturated or unsaturated C₇-C₂₁ hydrocarbon-based radicals;

r, n and p, which may be identical or different, are integers ranging from 2 to 6;

y is an integer ranging from 1 to 10;

x and z, which may be identical or different, are integers ranging from 0 to 10;

X⁻ is a simple or complex organic or inorganic anion;

with the proviso that the sum x+y+z is from 1 to 15, that when x is 0, then R₁₆ denotes R₂₀ and that when z is 0, then R₁₈ denotes R₂₂.

The alkyl radicals R₁₅ may be linear or branched, and more particularly linear. Preferably, R₁₅ denotes a methyl, ethyl, hydroxyethyl or dihydroxypropyl radical, and more particularly a methyl or ethyl radical.

Avantageously, the sum x+y+z is from 1 to 10. When R₁₆ is a hydrocarbon-based radical R₂₀, it may be long and contain from 12 to 22 carbon atoms, or short and contain from 1 to 3 carbon atoms. When R₁₈ is a hydrocarbon-based radical R₂₂, it preferably contains 1 to 3 carbon atoms.

Avantageously, R₁₇, R₁₉ and R₂₁, which may be identical or different, are chosen from linear or branched, saturated or unsaturated C₁₁-C₂₁ hydrocarbon-based radicals, and more particularly from linear or branched, saturated or unsaturated

C₁₁-C₂, alkyl and alkenyl radicals. Preferably, x and z, which may be identical or different, are 0 or 1.

Avantageously, y is equal to 1.

Preferably, r, n and p, which may be identical or different, are equal to 2 or 3 and even more particularly equal to 2.

The anion X is preferably a halide (chloride, bromide or iodide) or a C₁-C₄ alkyl sulfate, more particularly methyl sulfate. However, methanesulfonate, phosphate, nitrate, tosylate, an anion derived from an organic acid, such as acetate or lactate, or any other anion that is compatible with the ammonium containing an ester function may be used.

The anion X⁻ is even more particularly chloride or methyl sulfate. Use is made more particularly in the composition according to the invention of the ammonium salts of formula (IV) in which:

R₁₅ denotes a methyl or ethyl radical,

x and y are equal to 1;

z is equal to 0 or 1;

r, n and p are equal to 2;

R₁₆ is chosen from:

a radical

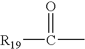

methyl, ethyl or C₁₄-C₂₂ hydrocarbon-based radicals,

a hydrogen atom;

R₁₈ is chosen from:

a radical

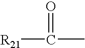

a hydrogen atom;

R₁₇, R₁₉ and R₂₁, which may be identical or different, are chosen from linear or branched, saturated or unsaturated C₁₃-C₁₇ hydrocarbon-based radicals, and preferably from linear or branched, saturated or unsaturated C₁₃-C₁₇ alkyl and alkenyl radicals. The hydrocarbon-based radicals are advantageously linear.

Examples of compounds of formula (VIII) that may be mentioned include the salts (especially chloride or methyl sulfate) of diacyloxyethyl-dimethylammonium, of diacyloxyethyl-hydroxyethyl-methylammonium, of monoacyloxyethyl-dihydroxyethyl-methylammonium, of triacyloxyethyl-methylammonium, of monoacyloxyethyl-hydroxyethyl-dimethylammonium, and mixtures thereof. The acyl radicals preferably contain 14 to 18 carbon atoms and are more particularly derived from a plant oil, for instance palm oil or sunflower oil. When the compound contains several acyl radicals, these radicals may be identical or different.

These products are obtained, for example, by direct esterification of optionally oxyalkylenated triethanolamine, triisopropanolamine, alkyldiethanolamine or alkyldiisopropanolamine onto fatty acids or onto mixtures of fatty acids of plant or animal origin, or by transesterification of the methyl esters thereof. This esterification is followed by a quaternization using an alkylating agent such as an alkyl halide (preferably a methyl or ethyl halide), a dialkyl sulfate (preferably dimethyl or diethyl sulfate), methyl methanesulfonate, methyl para-toluenesulfonate, glycol chlorohydrin or glycerol chlorohydrin.

Such compounds are sold, for example, under the names Dehyquart® by the company Cognis, Stepanquat® by the company Stepan, Noxamium® by the company Ceca, and Rewoquat® WE 18 by the company Rewo-Goldschmidt.

The composition according to the invention may preferably contain a mixture of quaternary ammonium mono-, di- and triester salts with a weight majority of diester salts.

Examples of mixtures of ammonium salts that may be used include the mixture containing 15% to 30% by weight of acyloxymethyl-dihydroxyethyl-methylammonium methyl sulfate, 45% to 60% of diacyloxyethyl-hydroxyethyl-methylammonium methyl sulfate and 15% to 30% of triacyloxyethyl-methylammonium methyl sulfate, the acyl radicals containing from 14 to 18 carbon atoms and being derived from optionally partially hydrogenated palm oil.

It is also possible to use the ammonium salts containing at least one ester function described in patents U.S. Pat. No. 4,874,554 and U.S. Pat. No. 4,137,180.

Among the quaternary ammonium salts mentioned above that are preferably used are those corresponding to formula (V). Mention may be made firstly of tetralkylammonium chlorides, for instance dialkyldimethylammonium or alkyl-trimethylammonium chlorides in which the alkyl radical contains from about 12 to 22 carbon atoms, in particular behenyltrimethylammonium, distearyldimethyl-ammonium, cetyltrimethylammonium or benzyldimethylstearylammonium chloride, or alternatively, secondly of palmitylamidopropyltrimethylammonium chloride or stearamidopropyldimethyl(myristyl acetate)ammonium chloride sold under the name Ceraphyl® 70 by the company Van Dyk.

The cationic surfactants that are particularly preferred in the composition of the invention are chosen from quaternary ammonium salts, and in particular from behenyltrimethylammonium chloride, cetyltrimethylammoniumchloride, quaternium-83, behenylamidopropyl-2,3-dihydroxypropyidimethylammonium chloride and palmitylamidopropyltrimethylammonium chloride.

The preferred cationic surfactants are cationic surfactants that are soluble in the composition and in particular those that are water-soluble, or those that are dissolved in water or in the composition by at least one nonionic surfactant.

The expression “cationic surfactants that are soluble in water or in the composition” means cationic surfactants that are soluble in water or in the composition at a concentration of greater than or equal to 0.1% by weight in water at 25° C., i.e. they form under these conditions a macroscopically isotropic transparent solution.

The composition according to the invention preferably contains the cationic surfactant(s) in an amount ranging from 0.05% to 10% by weight, preferably from 0.1% to 5% by weight, more particularly from 0.2% to 2% by weight and even more preferably from 0.3% to 1% by weight, relative to the total weight of the composition.

The compositions according to the invention also comprise at least two cationic polymers that are different than/from each other.

The cationic polymers that may be used in accordance with the present invention include those already known per se, and preferably include those known as improving cosmetic properties, i.e. especially those described in patent application EP-A-0 337 354 and in French patent applications FR-A-2 270 846, 2 383 660, 2 598 611, 2 470 596 and 2 519 863.

The cationic polymers that are preferred are chosen from those containing units comprising primary, secondary, tertiary and/or quaternary amine groups that may either form part of the main polymer chain or be borne by a side substituent directly attached thereto.

The cationic polymers used preferably have a number-average molecular mass (molecular weight) of between 500 and 5×10⁶ approximately and more preferably between 10³ and 3×10⁶ approximately.

Among the cationic polymers that may be mentioned more particularly are polymers of the polyamine, polyamino amide and polyquaternary ammonium type. These are known products.

The polymers of the polyamine, polyamino amide and polyquaternary ammonium type that may be used in accordance with the present invention, and that may especially be mentioned, are those described in French patents Nos 2 505 348 and 2 542 997. Among these polymers, particular mention may be made of:

(1) homopolymers or copolymers derived from acrylic or methacrylic esters or amides and comprising at least one of the units of the following formulae:

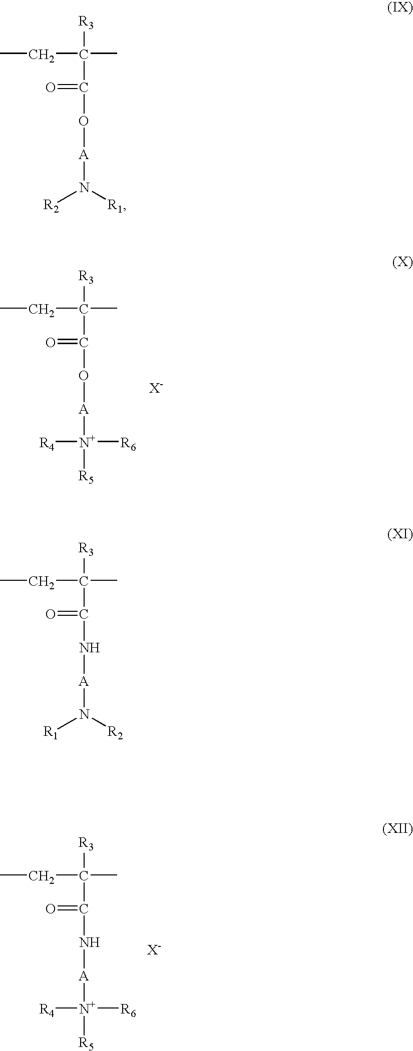

in which:

R₁ and R₂, which may be identical or different, represent hydrogen or an alkyl group containing from 1 to 6 carbon atoms, and preferably methyl or ethyl;

R₃, which may be identical or different, denote a hydrogen atom or a CH₃ radical;

A, which may be identical or different, represent a linear or branched alkyl group of 1 to 6 carbon atoms, preferably 2 or 3 carbon atoms, or a hydroxyalkyl group of 1 to 4 carbon atoms;

R₄, R₅, R₆, which may be identical or different, represent an alkyl group containing from 1 to 18 carbon atoms or a benzyl radical and preferably an alkyl group containing from 1 to 6 carbon atoms;

X⁻ denotes an anion derived from a mineral or organic acid, such as a methyl sulfate anion, an ethyl sulfate anion or a halide such as chloride or bromide.

The copolymers of family (1) can also contain one or more units derived from comonomers which may be chosen from the family of acrylamides, methacrylamides, diacetone acrylamides, acrylamides and methacrylamides substituted on the nitrogen with lower (C₁-C₄) alkyls, acrylic or methacrylic acids or esters thereof, vinyllactams such as vinylpyrrolidone or vinylcaprolactam, and vinyl esters.

Thus, among these copolymers of family (1), particular mention may be made of:

copolymers of acrylamide and of dimethylaminoethyl methacrylate quaternized with dimethyl sulfate or with a dimethyl halide, such as the product sold under the name Hercofloc by the company Hercules,

the copolymers of acrylamide and of methacryloyloxyethyltrimethylammonium chloride described, for example, in patent application EP-A-080 976 and sold under the name Bina Quat P 100 by the company Ciba,

the copolymer of acrylamide and of methacryloyloxyethyltrimethylammonium methosulfate sold under the name Reten by the company Hercules,

quaternized or nonquatemized vinylpyrrolidone/ dialkylaminoalkyl acrylate or methacrylate copolymers, such as the products sold under the name “Gafquat” by the company ISP, such as, for example, “Gafquat® 734” or “Gafquat® 755”, or alternatively the products known as “Copolymer 845, 958 and 937”. These polymers are described in detail in French patents 2 077 143 and 2 393 573,

dimethylaminoethyl methacrylate/vinylcaprolactam/vinylpyrrolidone terpolymers, such as the product sold under the name Gaffix® VC 713 by the company ISP,

vinylpyrrolidone/methacrylamidopropyldimethylamine copolymers sold in particular under the name Styleze® CC 10 by ISP,

quaternized vinylpyrrolidone/dimethylaminopropylmethacrylamide copolymers, such as the product sold under the name “Gafquat® HS 100” by the company ISP.

2) cationic polysaccharides, especially cationic celluloses, cationic starches and cationic galactomannan gums. Among the cationic polysaccharides that may be mentioned more particularly are cellulose ethers comprising quaternary ammonium groups, cationic cellulose copolymers or celluloses grafted with a water-soluble quaternary ammonium monomer and cationic galactomannan gums.

The cellulose ethers comprising quaternary ammonium groups, which are described in French patent 1 492 597 and in particular the polymers sold under the names “JR” (JR 400, JR 125, JR 30M) or “LR” (LR 400, LR 30M) by the company Amerchol. These polymers are also defined in the CTFA dictionary as hydroxyethylcellulose quaternary ammoniums that have reacted with an epoxide substituted with a trimethylammonium group.

The cationic cellulose copolymers or celluloses grafted with a water-soluble quaternary ammonium monomer are described especially in patent U.S. Pat. No. 4 131 576, such as hydroxyalkylcelluloses, for instance hydroxymethyl-, hydroxyethyl- or hydroxypropylcelluloses grafted especially with a methacryloyl-ethyltrimethylammonium, methacrylamidopropyltrimethylammonium or dimethyl-diallylammonium salt.

The commercial products corresponding to this definition are more particularly the products sold under the names Celquat®) L 200 and Celquat® H 100 by the company National Starch.

The cationic galactomannan gums are described more particularly in patents U.S. Pat. Nos. 3,589,578 and 4,031,307, in particular guar gums containing trialkyl-ammonium cationic groups. Use is made, for example, of guar gums modified with a salt (e.g. chloride) of 2,3-epoxypropyltrimethylammonium.

Such products are sold especially under the trade names Jaguar® C13 S, Jaguar® C 15, Jaguar® C 17 or Jaguar® C162 by the company Rhodia Chimie.

Starches modified with a 2,3-epoxypropyltrimethylammonium salt (e.g. chloride), for instance the product known as Starch hydroxypropyltrimonium chloride according to the INCl nomenclature and sold under the name Sensomer Cl-50 from Ondeo, may also be used.

(3) polymers consisting of piperazinyl units and of divalent alkylene or hydroxyalkylene radicals containing straight or branched chains, optionally interrupted by oxygen, sulfur or nitrogen atoms or by aromatic or heterocyclic rings, as well as the oxidation and/or quaternization products of these polymers. Such polymers are described, in particular, in French patents 2 162 025 and 2 280 361;

(4) water-soluble polyamino amides prepared in particular by polycondensation of an acidic compound with a polyamine; these polyamino amides can be crosslinked with an epihalohydrin, a diepoxide, a dianhydride, an unsaturated dianhydride, a bis-unsaturated derivative, a bis-halohydrin, a bis-azetidinium, a bis-haloacyldiamine, a bis-alkyl halide or alternatively with an oligomer resulting from the reaction of a difunctional compound which is reactive with a bis-halohydrin, a bis-azetidinium, a bis-haloacyldiamine, a bis-alkyl halide, an epihalohydrin, a diepoxide or a bis-unsaturated derivative; the crosslinking agent being used in proportions ranging from 0.025 to 0.35 mol per amine group of the polyamino amide; these polyamino amides can be alkylated or, if they contain one or more tertiary amine functions, they can be quaternized. Such polymers are described, in particular, in French patents 2 252 840 and 2 368 508;

(5) polyaminoamides resulting from the condensation of polyalkylene polyamines with polycarboxylic acids followed by alkylation with difunctional agents. Mention may be made, for example, of adipic acid/dialkylamino-hydroxyalkyldialkylenetriamine polymers in which the alkyl radical contains from 1 to 4 carbon atoms and preferably denotes methyl, ethyl or propyl. Such polymers are described in particular in French patent 1 583 363.

Among these derivatives, mention may be made more particularly of the adipic acid/dimethylaminohydroxypropyl/diethylenetriamine polymers sold under the name “Cartaretine® F, F4 or F8” by the company Sandoz.

(6) polymers obtained by reaction of a polyalkylene polyamine containing two primary amine groups and at least one secondary amine group with a dicarboxylic acid chosen from diglycolic acid and saturated aliphatic dicarboxylic acids having from 3 to 8 carbon atoms. The molar ratio between the polyalkylene polyamine and the dicarboxylic acid being between 0.8:1 and 1.4:1; the polyamino amide resulting therefrom being reacted with epichlorohydrin in a molar ratio of epichlorohydrin relative to the secondary amine group of the polyamino amide of between 0.5:1 and 1.8:1. Such polymers are described in particular in U.S. Pat. Nos. 3,227,615 and 2,961,347.

Polymers of this type are sold in particular under the name “Hercosett® 57” by the company Hercules Inc. or alternatively under the names “PD 170” or “Delsette® 101 ” by the company Hercules in the case of the adipic acid/epoxypropyl/diethylenetriamine copolymer.

(7) cyclopolymers of alkyldiallylamine or of dialkyidiallylammonium, such as the homopolymers or copolymers containing, as main constituent of the chain, units corresponding to formula (XIII) or (XIV):

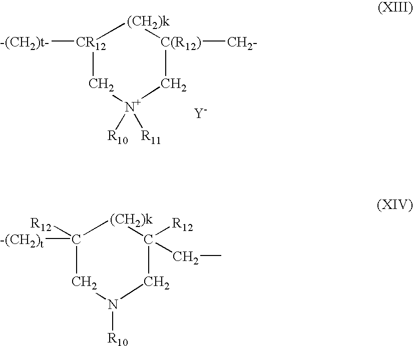

in which formulae k and t are equal to 0 or 1, the sum k+t being equal to 1; R₁₂ denotes a hydrogen atom or a methyl radical; R₁₀ and R₁₁, independently of each other, denote an alkyl group having from 1 to 8 carbon atoms, a hydroxyalkyl group in which the alkyl group preferably has 1 to 5 carbon atoms, a lower C₁-C₄ amidoalkyl group, or R₁₀ and R₁₁ can denote, together with the nitrogen atom to which they are attached, heterocyclic groups such as piperidyl or morpholinyl; Y⁻ is an anion such as bromide, chloride, acetate, borate, citrate, tartrate, bisulfate, bisulfite, sulfate or phosphate. These polymers are described in particular in French patent 2 080 759 and in its Certificate of Addition 2 190 406. R₁₀ and R₁₁, independently of each other, preferably denote an alkyl group containing from 1 to 4 carbon atoms.

Among the polymers defined above, mention may be made more particularly of the dimethyldiallylammonium chloride homopolymer sold under the name Merquat® 100 by the company Nalco (its homologues of low weight-average molecular mass) and copolymers of diallyldimethylammonium chloride and of acrylamide, sold under the name Merquat®) 550.

(8) quaternary diammonium polymers containing repeating units corresponding to the formula:

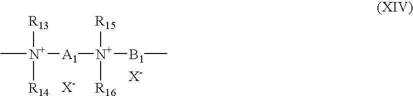

in which formula (XIV):

R₁₃, R₁₄, R₁₅ and R₁₆, which may be identical or different, represent aliphatic, alicyclic or arylaliphatic radicals containing from 1 to 20 carbon atoms or lower hydroxyalkylaliphatic radicals, or alternatively R₁₃, R₁₄, R₁₅ and R₁₆, together or separately, constitute, with the nitrogen atoms to which they are attached, heterocycles optionally containing a second hetero atom other than nitrogen, or alternatively R₁₃, R₁₄, R₁₅ and R₁₆ represent a linear or branched C₁-C₆ alkyl radical substituted with a nitrile, ester, acyl or amide group or a group —CO—O—R₁₇—D or —CO—NH—R₁₇—D where R₁₇ is an alkylene and D is a quaternary ammonium group;

A₁ and B₁ represent polymethylene groups containing from 2 to 20 carbon atoms, which groups may be linear or branched, saturated or unsaturated, and which may contain, linked to or intercalated in the main chain, one or more aromatic rings or one or more oxygen or sulfur atoms or sulfoxide, sulfone, disulfide, amino, alkylamino, hydroxyl, quaternary ammonium, ureido, amide or ester groups, and

X⁻ denotes an anion derived from an inorganic or organic acid;

A₁, R₁₃ and R₁₅ can form, with the two nitrogen atoms to which they are attached, a piperazine ring; in addition, if A₁ denotes a linear or branched, saturated or unsaturated alkylene or hydroxyalkylene radical, B₁ can also denote a group (CH₂)ₙ—CO—D—OC—(CH₂)ₙ—

in which D denotes:

a) a glycol residue of formula: —O-Z-O—, where Z denotes a linear or branched hydrocarbon-based radical or a group corresponding to one of the following formulae:

—(CH₂—CH₂—O)ₓ—CH₂—CH₂—

—[CH₂—CH(CH₃)—O]_{y}—CH₂—CH(CH₃)—

where x and y denote an integer from 1 to 4, representing a defined and unique degree of polymerization or any number from 1 to 4 representing an average degree of polymerization;

b) a bis-secondary diamine residue such as a piperazine derivative;

c) a bis-primary diamine residue of formula: —NH—Y—NH—, where Y denotes a linear or branched hydrocarbon-based radical, or alternatively the divalent radical

—CH₂—CH₂—S—S—CH₂—CH₂—;

d) a ureylene group of formula: —NH—CO—NH—;

Preferably, X⁻ is a monovalent mineral or organic anion such as a halide (chloride or bromide), a sulfate or a carboxylate (acetate, lactate or citrate).

These polymers generally have a number-average molecular mass (molecular weight) of between 1 000 and 100 000.

Polymers of this type are described in particular in French patents 2 320 330, 2 270 846, 2 316 271, 2 336 434 and 2 413 907 and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945 and 4,027,020.

It is more particularly possible to use polymers which consist of repeating units corresponding to the formula:

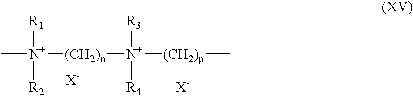

in which R₁, R₂, R₃ and R₄, which may be identical or different, denote an alkyl or hydroxyalkyl radical containing from 1 to 4 carbon atoms approximately, n and p are integers ranging from 2 to 20 approximately, and X is an anion derived from an inorganic or organic acid.

One compound of formula (XV) which is particularly preferred is the one for which R₁, R₂, R₃ and R₄ represent a methyl radical and n=3, p=6 and X=Cl, which is known as Hexadimethrine chloride according to the INCl (CTFA) nomenclature.

(9) polyquaternary ammonium polymers consisting of units of formula (XVI):

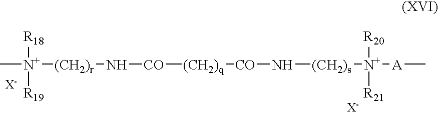

in which formula:

R₁₈, R₁₉, R₂₀ and R₂₁, which may be identical or different, represent a hydrogen atom or a methyl, ethyl, propyl, β-hydroxyethyl, β-hydroxypropyl or —CH₂CH₂(OCH₂CH₂)ₚOH radical,

where p is equal to 0 or to an integer between 1 and 6, with the proviso that R₁₈, R₁₉, R₂₀ and R₂₁ do not simultaneously represent a hydrogen atom,

r and s, which may be identical or different, are integers between 1 and 6,

q is equal to 0 or to an integer between 1 and 34,

X⁻ denotes an anion such as a halide,

A denotes a dihalide radical or preferably represents —CH₂—CH₂—O—CH₂—CH₂—.

Such compounds are described in particular in patent application EP-A-122 324. Among these products, mention may be made, for example, of “Mirapol® A 15”, “Mirapol® AD1”, “Mirapol® AZ1 ” and “Mirapol®175” sold by the company Miranol.

(10) quaternary polymers of vinylpyrrolidone and of vinylimidazole, such as polyquaternium-11, polyquaternium-16 and polyquaternium-44, especially the products sold under the names Luviquat® FC 905, FC 550, FC 370 and Luviquat® Care by the company BASF.

(11) polyamines, for instance Polyquart® H sold by Cognis, referenced under the name Polyethylene Glycol (15) Tallow Polyamine in the CTFA dictionary.

(12) Crosslinked or noncrosslinked methacryloyloxy(C₁-C₄)alkyltri(C₁-C₄)alkyl-ammonium salt polymers such as the polymers obtained by homopolymerization of dimethylaminoethyl methacrylate quaternized with methyl chloride, or by copolymerization of acrylamide with dimethylaminoethyl methacrylate quaternized with methyl chloride, the homo- or copolymerization being followed by crosslinking with a compound containing olefinic unsaturation, in particular methylene bisacrylamide. A crosslinked acrylamide/methacryloyloxyethyltrimethylammonium chloride copolymer (20/80 by weight) in the form of a dispersion containing 50% by weight of said copolymer in mineral oil can be used more particularly. This dispersion is sold under the name “Salcare® SC 92” by the company Ciba. A crosslinked methacryloyloxyethyltrimethylammonium chloride homopolymer containing about 50% by weight of the homopolymer in mineral oil or in a liquid ester can also be used. These dispersions are sold under the names “Salcare® SC 95” and “Salcare® SC 96” by the company Ciba.

Other cationic polymers that can be used in the context of the invention include cationic proteins or cationic protein hydrolyzates, polyalkyleneimines, in particular polyethyleneimines, polymers containing vinylpyridine or vinylpyridinium units, condensates of polyamines and of epichlorohydrin, quaternary polyureylenes and chitin derivatives.

Among all the cationic polymers that may be used in the context of the present invention, it is preferred to use the cationic polysaccharides, in particular quaternary cellulose ether derivatives such as the products sold under the name “JR 400” by the company Amerchol, cationic cyclopolymers, in particular the dimethyldiallylammonium chloride homopolymers or copolymers sold under the names “Merquat® 100”, “Merquat® 550” and “Merquat® S” by the company Nalco, copolymers of vinylpyrrolidone and of methylvinylimidazolium salts (e.g. methyl sulfate or ethyl sufate) sold especially under the name Luviquat Care by BASF, and mixtures thereof.

According to the invention, the composition preferably comprises at least one cationic polysaccharide and at least one vinylpyrrolidone copolymer, in particular quaternary copolymers of vinylpyrrolidone and of vinylimidazole.

According to the invention, the composition preferably comprises at least one diallyldimethylammonium chloride polymer, preferably a homopolymer, and at least one vinylpyrrolidone copolymer, in particular quaternary copolymers of vinylpyrrolidone and of vinylimidazole.

According to the invention, each cationic polymer may preferably represent from 0.001% to 20% by weight, more preferably from 0.01% to 10% by weight, even more preferably from 0.05% to 5% by weight and more particularly from 0.1% to 3% by weight, and even more particularly from 0.1% to 1% by weight, relative to the total weight of the final composition.

According to the invention, the total concentration of cationic polymer may preferably represent from 0.01% to 20% by weight, more preferably from 0.05% to 5% by weight, more particularly from 0.1% to 3% by weight and even more particularly from 0.5% to 1.5% by weight, relative to the total weight of the final composition.

According to the invention, the expression “nonassociative thickening polymer” means a thickening polymer not simultaneously comprising at least one C8-C30 fatty chain and at least one hydrophilic unit.

The nonionic nonassociative thickening polymers according to the invention may be of natural or synthetic origin. They include and are chosen especially from:

(i) nonionic homopolymers and copolymers containing ethylenically unsaturated monomers of ester and/or amide type,

(ii) vinylpyrrolidone homopolymers or copolymers,

(iii) polysaccharides.

Among the nonionic homopolymers or copolymers containing ethylenically unsaturated monomers of ester and/or amide type that may be mentioned are polyamides, especially the products sold under the names: Cyanamer P250 by the company Cytec (polyacrylamide); methyl methacrylate/ethylene glycol dimethacrylate copolymers (PMMA MBX-8C by the company US Cosmetics); butyl methacrylate/methyl methacrylate copolymers (Acryloid B66 by the company Rohm & Haas); polymethyl methacrylate (BPA 500 by the company Kobo).

The vinylpyrrolidone homopolymers or copolymers are chosen especially from crosslinked vinylpyrrolidone homopolymers such as the Polymer ACP-10 sold by ISP.

The thickening polysaccharides are especially chosen from glucans, modified or unmodified starches (such as those derived, for example, from cereals, for instance wheat, corn or rice, from vegetables, for instance yellow pea, and tubers, for instance potato or cassava), amylose, amylopectin, glycogen, dextrans, celluloses and derivatives thereof (methylcelluloses, hydroxyalkylcelluloses, ethylhydroxyethylcelluloses), mannans, xylans, lignins, arabans, galactans, galacturonans, chitin, chitosans, glucuronoxylans, arabinoxylans, xyloglucans, glucomannans, pectic acids and pectins, arabinogalactans, carrageenans, agars, gum arabics, gum tragacanths, ghatti gums, karaya gums, carob gums, galacto-mannans such as guar gums and nonionic derivatives thereof (hydroxypropyl guar), and mixtures thereof.

In general, the compounds of this type that may preferably be used in the present invention are chosen from those described especially in “Encyclopedia of Chemical Technology”, Kirk-Othmer, Third Edition, 1982, volume 3, pp. 896-900, and volume 15, pp. 439-458, in “Polymers in Nature” by E. A. MacGregor and C. T. Greenwood, published by John Wiley & Sons, Chapter 6, pp. 240-328,1980, and in “Industrial Gums—Polysaccharides and their Derivatives”, edited by Roy L. Whistler, Second Edition, published by Academic Press Inc., the content of these three publications being entirely included in the present patent application by way of reference.

Starches, guar gums and celluloses and derivatives thereof will preferably be used.

The polysaccharides can be modified or unmodified. The unmodified guar gums include, for example, the products sold under the name Vidogum GH 175 by the company Unipectine and under the names Meypro-Guar 50 and Jaguar C by the company Rhodia Chimie.

The modified nonionic guar gums are especially modified with C₁-C₆ hydroxyalkyl groups. Among the hydroxyalkyl groups that may be mentioned, for example, are hydroxymethyl, hydroxyethyl, hydroxypropyl and hydroxybutyl groups. These guar gums are well known in the prior art and can be prepared, for example, by reacting the corresponding alkene oxides such as, for example, propylene oxides, with the guar gum so as to obtain a guar gum modified with hydroxypropyl groups.

The degree of hydroxyalkylation, which corresponds to the number of alkylene oxide molecules consumed by the number of free hydroxyl functions present on the guar gum, preferably ranges from 0.4 to 1.2.

Such nonionic guar gums optionally modified with hydroxyalkyl groups are sold, for example, under the trade names Jaguar HP8, Jaguar HP60 and Jaguar HP120, Jaguar DC 293 and Jaguar HP 105 by the company Rhodia Chimie or under the name Galactasol 4H4FD2 by the company Aqualon.

Among the celluloses that are especially used are hydroxyethylcelluloses and hydroxypropylcelluloses. Mention may be made of the products sold under the names Klucel EF, Klucel H, Klucel LHF, Klucel MF and Klucel G by the company Aqualon, and Cellosize Polymer PCG-10 by the company Amerchol.

Preferably, the thickening polymers in the cosmetic compositions in accordance with the present invention advantageously have in solution or in dispersion, at a concentration of 1% acitve material in water, a viscosity measured using a Brookfield viscometer at a shear rate of 20 T/min, of greater than 5 mPa/s and even more advantageously greater than 10 mPa/s.

According to the invention, the thickening polymer(s) preferably represent from 0.001% to 20% by weight, more preferably from 0.01% to 10% by weight, more particularly from 0.1% to 3% by weight and even more particularly from 0.2% to 2% by weight, relative to the total weight of the final composition.

The composition according to the invention may optionally contain surfactants other than cationic surfactants. The surfactants that may be used in the present invention include those chosen from the anionic, nonionic and amphoteric surfactants that are well known in the art, and mixtures thereof.

The surfactants are generally preferably present in an amount of between 0.1% and 10% by weight approximately, more preferably between 0.5% and 8% and even more preferably between 1% and 5%, relative to the total weight of the composition.

The composition according to the invention preferably contains at least one surfactant chosen from nonionic surfactants.

The nonionic surfactants include, themselves also, compounds that are well known per se (see in particular in this respect “Handbook of Surfactants” by M. R. Porter, published by Blackie & Son (Glasgow and London), 1991, pp. 116-178) and, in the context of the present invention, their nature is not a critical feature. Thus, they can be chosen in particular from (non-limiting list) polyethoxylated, polypropoxylated or polyglycerolated fatty acids, alkylphenols, α-diols or alcohols having a fatty chain containing, for example, 8 to 18 carbon atoms, it being possible for the number of ethylene oxide or propylene oxide groups to range in particular from 2 to 50 and for the number of glycerol groups to range in particular from 2 to 30. Mention may also be made of copolymers of ethylene oxide and of propylene oxide, condensates of ethylene oxide and of propylene oxide with fatty alcohols; polyethoxylated fatty amides preferably having from 2 to 30 mol of ethylene oxide, polyglycerolated fatty amides containing on average 1 to 5, and in particular 1.5 to 4, glycerol groups; oxyethylenated fatty acid esters of sorbitan having from 2 to 30 mol of ethylene oxide; fatty acid esters of sucrose, fatty acid esters of polyethylene glycol, alkylpolyglycosides, N-alkylglucamine derivatives, amine oxides such as (C₁₀-C₁₄)alkylamine oxides or N-acylaminopropylmorpholine oxides. It will be noted that the alkylpolyglycosides constitute nonionic surfactants that are particularly suitable in the context of the present invention.

The concentration of nonionic surfactants in the nondetergent compositions preferably ranges from 0.1% to 10% by weight, more preferably from 0.5% to 8% by weight and more particularly from 1% to 5% by weight relative to the total weight of the composition.

The nonwashing (nondetergent) compositions preferably comprise less than 3% by weight of detergent surfactants, especially anionic detergent surfactants, and more particularly less than 1% by weight, relative to the total weight of the composition.

The nonwashing (nondetergent) compositions preferably comprise less than 3% by weight of anionic surfactants and more particularly less than 1% by weight, relative to the total weight of the composition.

The composition according to the invention may also comprise at least one conditioner chosen from silicones other than silicones containing a quaternary ammonium group, carboxylic esters containing at least 12 carbon atoms, plant oils, mineral oils and synthetic oils such as poly(α-olefins), and mixtures thereof.

The silicones that may be used in accordance with the invention may be soluble or insoluble in the composition, and they may be in particular polyorganosiloxanes that are insoluble in the composition of the invention. They may be in the form of oils, waxes, resins or gums. They may be used pure or as an emulsion, a dispersion or a microemulsion.

The organopolysiloxanes are defined in greater detail in Walter Noll's “Chemistry and Technology of Silicones” (1968) Academic Press. They can be volatile or nonvolatile.

When they are volatile, the silicones are more particularly chosen from those having a boiling point of between 60° C. and 260° C., and even more particularly from:

(i) cyclic silicones containing from 3 to 7 and preferably 4 to 5 silicon atoms.

These are, for example, octamethylcyclotetrasiloxane sold in particular under the name “Volatile Silicone 7207” by Union Carbide or “Silbione 70045 V 2” by Rhodia, decamethylcyclopentasiloxane sold under the name 'Volatile Silicone 7158” by Union Carbide, and “Silbione 70045 V 5” by Rhodia, and mixtures thereof.

Mention may also be made of cyclocopolymers of the dimethylsiloxane/methylalkylsiloxane type, such as “Silicone Volatile FZ 3109” sold by the company Union Carbide, having the chemical structure:

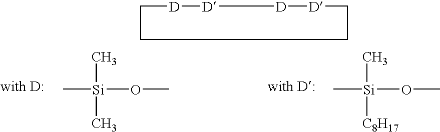

Mention may also be made of mixtures of cyclic silicones with organosilicon compounds, such as the mixture of octamethylcyclotetrasiloxane and tetratrimethylsilylpentaerythritol (50/50) and the mixture of octamethylcyclo-tetrasiloxane and oxy-1,1′-bis(2,2,2′,2′,3,3′-hexatrimethylsilyloxy)neopentane;

(ii) linear volatile silicones containing 2 to 9 silicon atoms and having a viscosity of less than or equal to 5×10⁻⁶ m²/s at 25° C. An example is decamethyltetrasiloxane sold in particular under the name “SH 200” by the company Toray Silicone. Silicones belonging to this category are also described in the article published in Cosmetics and Toiletries, Vol. 91, Jan. 76, pp. 27-32, Todd & Byers “Volatile Silicone Fluids for Cosmetics”.

Among the nonvolatile silicones that may especially be mentioned are polyalkyl-siloxanes, polyarylsiloxanes, polyalkylarylsiloxanes, silicone gums and resins, polyorganosiloxanes modified with organofunctional groups, and also mixtures thereof.

The organomodified silicones that can be used in accordance with the invention are silicones as defined above and containing in their structure one or more organofunctional groups attached via a hydrocarbon-based group.

Among the organomodified silicones, mention may be made of polyorganosiloxanes comprising:

polyethyleneoxy and/or polypropyleneoxy groups optionally comprising C₆-C₂₄ alkyl groups, such as the products known as dimethicone copolyol sold by the company Dow Corning under the name DC 1248 or the oils Silwet® L 722, L 7500, L 77 and L 711 by the company Union Carbide, and the (C₁₂)alkylmethicone copolyol sold by the company Dow Corning under the name Q2 5200;

substituted or unsubstituted amine groups, such as the products sold under the name GP 4 Silicone Fluid and GP 7100 by the company Genesee, or the products sold under the names Q2 8220 and Dow Corning 929 or 939 by the company Dow Corning. The substituted amine groups are, in particular, C₁-C₄ aminoalkyl groups;

thiol groups such as the products sold under the names “GP 72 A” and “GP 71” from Genesee;

alkoxylated groups such as the product sold under the name “Silicone Copolymer F-755” by SWS Silicones and Abil Wax® 2428, 2434 and 2440 by the company Goldschmidt;

hydroxylated groups such as the polyorganosiloxanes containing a hydroxyalkyl function, described in French patent application FR-A-85/16334;

acyloxyalkyl groups such as, for example, the polyorganosiloxanes described in patent U.S. Pat. No. 4,957,732;

anionic groups of the carboxylic acid type, such as, for example, in the products described in patent EP 186 507 from the company Chisso Corporation, or of the alkylcarboxylic type, such as those present in the product X-22-3701 E from the company Shin-Etsu; 2-hydroxyalkyl sulfonate; 2-hydroxyalkyl thiosulfate such as the products sold by the company Goldschmidt under the names “Abil® S201” and “Abile S255”;

hydroxyacylamino groups, such as the polyorganosiloxanes described in patent application EP 342 834. Mention may be made, for example, of the product Q2-8413 from the company Dow Corning.

Examples of silicones that are preferably used include polydimethylsiloxanes, polyalkylarylsiloxanes and polydimethylsiloxanes containing amino or alkoxylated groups.

The composition according to the invention may also comprise one or more carboxylic acid esters, for instance compounds of formula RₐCOOR_{b} in which Rₐ represents a higher fatty acid residue containing from 4 to 29 carbon atoms and R_{b} represents a hydrocarbon-based chain containing from 3 to 30 carbon atoms, such as purcellin oil (stearyl octanoate), isopropyl myristate, isopropyl palmitate, butyl stearate, hexyl laurate, diisopropyl adipate, isononyl isononanoate, 2-ethylhexyl palmitate, 2-hexyldecyl laurate, 2-octyldecyl palmitate, 2-octyldodecyl myristate or lactate, and isostearyl neopentanoate, and mixtures thereof.

The composition according to the invention may also comprise one or more plant oils such as sweet almond oil, avocado oil, castor oil, olive oil, jojoba oil, sunflower oil, wheatgerm oil, sesame oil, groundnut oil, grapeseed oil, soybean oil, rapeseed oil, safflower oil, coconut oil, maize oil, hazelnut oil, karite butter, palm oil, apricot kernel oil and beauty-leaf oil, and mixtures thereof.

Mineral oils that may especially be mentioned include liquid paraffin and liquid petroleum jelly.

The conditioners, chosen from silicones, esters, plant oils, mineral oils and synthetic oils, and mixtures thereof, are preferably contained in the composition according to the invention in an amount ranging from 0.01% to 20% by weight, better still ranging from 0.1% to 10% by weight and more particularly ranging from 0.3% to 5% by weight relative to the total weight of the composition.

The (cosmetically acceptable) medium is preferably aqueous and may comprise water or a mixture of water and a cosmetically acceptable solvent such as a C₁-C₄ lower alcohol, for example ethanol, isopropanol, tert-butanol or n-butanol; polyols, for instance propylene glycol or glycerol; polyol ethers; C₅-C₁₀ alkanes; acetone or methyl ethyl ketone; C₁-C₄ alkyl acetates, for instance methyl acetate, ethyl acetate or butyl acetate; dimethoxyethane or diethoxyethane; and mixtures thereof. The solvents are preferably chosen from glycerol and propylene glycol.

The (cosmetically acceptable) medium, which is especially aqueous, preferably represents from 30% to 98% by weight relative to the total weight of the composition.

The solvents are preferably present in concentrations ranging from 0.5% to 30% by weight relative to the total weight of the composition.

The pH of the compositions of the invention is preferably between 2 and 8, and preferably between 3 and 7.

According to the invention, the compositions are preferably transparent. The transparency may be measured by means of the tubidity using a Hach Model 2100 P turbidimeter at 25° C. (the machine is calibrated with formazine). The turbidity of the compositions according to the invention (in the absence of additional insoluble compounds) is then generally between 0.05 and 500 NTU and preferably between 10 and 300 NTU.

The compositions according to the invention may also contain additives that are well known in the art, such as anionic, nonionic or amphoteric polymers, nonpolymeric thickeners, for instance acids or electrolytes, opacifiers, nacreas agents, vitamins, provitamins such as panthenol, waxes such as plant waxes, natural or synthetic ceramides, fragrances, colorants, organic or mineral particles, preserving agents and pH stabilizers.

A person skilled in the art knows how to select the optional additives and the amount thereof such that they do not harm the properties of the compositions of the present invention in view of this disclosure.

These additives may be present in the composition according to the invention in an amount ranging from 0% to 20% by weight relative to the total weight of the composition.

The compositions of the invention may be in any form including the form of a rinse-out or leave-in conditioner, or permanent waving, relaxing, dyeing or bleaching compositions, or alternatively in the form of rinse-out compositions to be applied before a dyeing, bleaching, permanent-waving or relaxing operation or alternatively between the two steps of a permenant-waving or relaxing operation.

They may be used, for example, as conditioners, care products deep-down care masks or scalp treatment lotions or creams. These compositions may be rinse-out or leave-in compositions.

According to one preferred embodiment of the invention, the composition may be used as a conditioner, in particular on fine hair. This conditioner may be a rinse-out or leave-in conditioner and preferably a rinse-out conditioner.

The (cosmetic) compositions according to the invention may be in the form of a gel, a milk, a cream, an emulsion, fluid or thickened lotions or a foam, and may be used for the skin, the nails, the eyelashes, the lips and, more particularly, the hair.

The compositions may be packaged in various forms, especially in vaporizers, pump-dispenser bottles or in aerosol containers in order to dispense the composition in vaporized form or in the form of a mousse. Such packaging forms are indicated, for example, when it is desired to obtain a spray, a lacquer or a mousse for treating the hair.

The present invention also relates to a (cosmetic) process for treating keratin materials such as, for example, the skin or the hair, which comprises applying an effective amount of a (cosmetic) composition as described above to the keratin materials, and optionally rinsing it off after optionally leaving it to act for a period of time. The rinsing may be performed, for example, with water.

Thus, this process according to the invention allows holding of the hairstyle and treatment, conditioning, or care of the hair or any other keratin material.

The examples that follow illustrate the present invention and should not in any way be considered as limiting the invention.

### EXAMPLE 1

The rinse-out conditioning composition below was prepared:

| Cetyltrimethylammonium chloride | 0.8 | g AM |
|---|---|---|
| (Dehyquart A OR from Cognis) | | |
| Quaternium-80 as a solution containing | 0.5 | g AM |
| 50% AM in propylene glycol | | |
| (Abil Quat 3272 from Goldschmidt) | | |
| Hydroxypropyl guar (Jaguar HP 105 from Rhodia Chimie) | 0.4 | g |
| Polyquaternium-10 (JR400 from Rhodia Chimie) | 0.5 | g |
| Polyquaternium-44 (Luviquat Care from BASF) | 0.57 | g AM |
| Hydroxyethylcellulose (Cellosize Polymer PCG-10 | 0.3 | g |
| from Union Carbide) | | |
| Glycerol | 5 | g |
| Oxyethylenated (20 EO) sorbitan monolaurate | 0.4 | g |
| (Tween 20 from Uniqema) | | |
| Citric acid | 0.4 | g |
| Fragrance | qs | |
| Preserving agents | qs | |
| Water qs | 100 | g |

The water at room temperature is introduced into a manufacturing tank, followed by addition of the preserving agents and the polyquaternium-10. The mixture is homogenized until dissolution is complete. Next, the hydroxypropyl guar, the hydroxyethylcellulose predispersed in the glycerol, and then the citric acid, the cetyltrimethylammonium chloride, the polyquaternium-44 and the quaternium-80 are successively added, with homogenization between each addition. When the mixture is homogeneous, the fragrance predispersed in the oxyethylenated (20 EO) sorbitan monolaurate is added.

The composition was applied for 1 to 5 minutes to washed and drained hair. The hair was then rinsed and dried.

The wet hair is then smooth and supple, and the dried hair is maleable and individualized.

### EXAMPLE 2

The rinse-out conditioning composition below was prepared:

| Cetyltrimethylammonium chloride | 0.8 | g AM |
|---|---|---|
| (Dehyquart A OR from Cognis) | | |
| Quaternium-80 as a solution containing | 0.5 | g AM |
| 50% AM in propylene glycol | | |
| (Abil Quat 3272 from Goldschmidt) | | |
| Hydroxypropyl guar (Jaguar HP 105 from Rhodia Chimie) | 0.2 | g |
| Polyquaternium-10 (JR400 from Rhodia Chimie) | 0.5 | g |
| Polyquaternium-44 (Luviquat Care from BASF) | 0.57 | g AM |
| Hydroxyethylcellulose (Cellosize Polymer PCG-10 | 0.7 | g |
| from Union Carbide) | | |
| Glycerol | 5 | g |
| Proylene glycol | 2 | g |
| Citric acid | 0.5 | g |
| Fragrance | qs | |
| Preserving agents | qs | |
| Water qs | 100 | g |

The composition was applied for 1 to 5 minutes to washed and drained hair. The hair was then rinsed and dried.

The wet hair is then smooth and supple, and the dried hair is shiny, supple and individualized.

### EXAMPLE 3

The rinse-out conditioning composition below was prepared:

| Cetyltrimethylammonium chloride | 0.8 | g AM |
|---|---|---|
| (Dehyquart A OR from Cognis) | | |
| Quaternium-80 as a solution containing | 0.5 | g AM |
| 50% AM in propylene glycol | | |
| (Abil Quat 3272 from Goldschmidt) | | |
| Hydroxypropyl guar (Jaguar HP 105 from Rhodia Chimie) | 0.2 | g |
| Polyquaternium-10 (JR400 from Rhodia Chimie) | 0.5 | g |
| Starch hydroxypropyltrimonium chloride | 0.62 | g AM |
| (Sensomer Cl-50 from Ondeo) | | |
| Hydroxyethylcellulose (Cellosize Polymer PCG-10 | 0.7 | g |
| from Union Carbide) | | |
| Glycerol | 5 | g |
| Proylene glycol | 2 | g |
| Citric acid | 0.5 | g |
| Fragrance | qs | |
| Preserving agents | qs | |
| Water qs | 100 | g |

The composition was applied for 1 to 5 minutes to washed and drained hair. The hair was then rinsed and dried.

The wet hair is then smooth and supple, and the dried hair is shiny, supple and individualized.

### EXAMPLE 4

The rinse-out conditioning composition below was prepared:

| Behenylamidopropyl-2,3- | 0.42 | g AM |
|---|---|---|
| dihydroxypropyldimethylammonium chloride | | |
| (Lexquat AMG-BEO from Inolex) | | |
| Quaternium-80 as a solution containing | 0.25 | g AM |
| 50% AM in propylene glycol | | |
| (Abil Quat 3272 from Goldschmidt) | | |
| Hydroxypropyl guar (Jaguar HP 105 from Rhodia Chimie) | 0.4 | g |
| Polyquaternium-10 (JR400 from Rhodia Chimie) | 0.5 | g |
| Polyquaternium-44 (Luviquat Care from BASF) | 0.57 | g AM |
| Hydroxyethylcellulose (Cellosize Polymer PCG-10 | 0.3 | g |
| from Union Carbide) | | |
| Glycerol | 5 | g |
| Citric acid | 0.2 | g |
| Fragrance | qs | |
| Preserving agents | qs | |
| Water qs | 100 | g |

The composition was applied for 1 to 5 minutes to hair washed and drained for 1 to 5 minutes. The hair was then rinsed and dried.

The wet hair is then smooth and supple, and the dried hair is shiny, supple and individualized.

As used herein, the terms “about” and “approximately” preferably mean +/−10%. The phrases “between X and Y” and “ranging from X to Y” include X and Y.

The above description of the invention sets forth the manner and process of making and using it such that it enables any person skilled in this art to make and use the same, specifically including the making and using of the following preferred embodiments and those set out in the claims, all of which make up a part of this description:

A nonwashing (cosmetic) composition comprising, preferably in a (cosmetically acceptable) medium, at least one silicone containing quaternary ammonium groups, at least one cationic surfactant, at least two different cationic polymers and at least one nonionic and nonassociative thickening polymer, and

methods of using the invention compositions to treat hair, skin, etc.

All references, documents, brochures, texts, articles, patents, applications, etc. mentioned above are incorporated herein by reference. All available literature for those commercially available materials mentioned herein is also incorporated herein by reference. Where a numerical limit or range is stated all endpoints are included, and all values and subranges within the stated ranges or limits are expressly included as if specifically written out.

1. A nonwashing composition comprising water, at least one silicone containing quaternary ammonium groups, at least one cationic surfactant, at least two different cationic polymers, and at least one nonionic and nonassociative thickening polymer.

2. The composition as claimed in claim 1, wherein said silicone containing quaternary ammonium groups is selected from the group consisting of silicones corresponding to one of the following formulae: 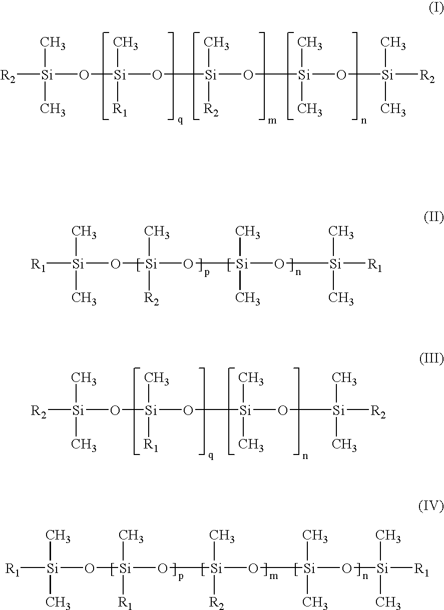
in which formulae:
R₁, which may be identical or different, represents a C₁-C₃₀ linear or branched alkyl group, or phenyl;
R₂, which may be identical or different, represents —C_{c}H_{2c}—O—(C₂H₄O)ₐ—(C₃H₆O)_{b}—R₅ or —C_{c}H_{2c}—O—(C₄H₈O)ₐ—R₅;
R₅, which may be identical or different, is selected from the group consisting of the groups having the following formula: 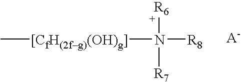
the radicals R₈ independently represent a linear or branched C₁₋₂₂ alkyl or C₂₋₂₂ alkenyl radical optionally bearing one or more OH groups, or represent a group CₕH₂ₕZCOR₉;
R₆, R₇ and R₉, which may be identical or different, represent linear or branched C₁₋₂₂ alkyl or C₂₋₂₂ alkenyl radicals optionally bearing one or more OH groups, or R₇ may form with a portion of R₈ a heterocycle;
m ranges from 0 to 20;
n ranges from 0 to 500;
p ranges from 1 to 50;
q ranges from 0 to 20;
a ranges from 0 to 50;
b ranges from 0 to 50;
c ranges from 0 to 4;
f ranges from 0 to 4;
g ranges from 0 to 2;
h ranges from 1 to 4;
Z represents an oxygen atom or NH,
A⁻ represents a monovalent mineral or organic anion such as a halide, a sulfate or a carboxylate.

3. The composition as claimed in claim 2, wherein said silicone is of formula (III).

4. The composition as claimed in claim 3, wherein said silicone corresponds to formula (III) in which at least one of the following conditions is met:
c is equal to 2 or 3;
R₁ denotes a methyl group;
a and b are equal to zero;
n ranges from 0 to 100;
q is equal to 0;
f=3;
g=1;
R₆ and R₇ denote a methyl group;
R₈ denotes a radical —(CH₂)—NHCOR₉.

5. The composition as claimed in claim 1, wherein said silicone is Quaternium-80.

6. The composition as claimed in claim 1, wherein the silicone containing quaternary ammonium groups is provided in the form of a solution, a suspension or a dispersion in water.

7. The composition as claimed in claim 1, which contains the silicone containing quaternary ammonium groups in an amount of from 0.01% to 10% by weight relative to the total weight of the composition.

8. The composition as claimed in claim 1, wherein the cationic surfactant is selected from the group consisting of optionally polyoxyalkylenated primary, secondary or tertiary fatty amine salts, quaternary ammonium salts, and mixtures thereof.

9. The composition as claimed in claim 8, comprising at least one quaternary ammonium salt selected from the group consisting of:
those of general formula (V) below: 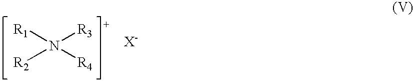
in which the symbols R₁ to R₄, which may be identical or different, represent a linear or branched aliphatic radical containing from 1 to 30 carbon atoms, or an aromatic radical such as aryl or alkylaryl; X is an anion selected from the group consisting of the group of halides, phosphates, acetates, lactates, (C₂-C₆) alkyl sulfates and alkyl- or alkylaryl-sulfonates;
quaternary ammonium salts of imidazoline;
diquaternary ammonium salts of formula (VII): 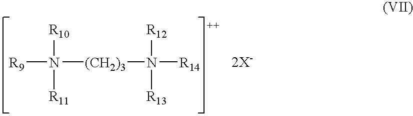
in which R₉ denotes an aliphatic radical containing from about 16 to 30 carbon atoms, R₁₀, R₁₁, R₁₂, R₁₃ and R₁₄, which may be identical or different, are selected from the group consisting of hydrogen and an alkyl radical containing from 1 to 4 carbon atoms, and X⁻ is an anion selected from the group consisting of the group of halides, acetates, phosphates, nitrates and methyl sulfates;
quaternary ammonium salts containing at least one ester function.

10. The composition as claimed in claim 9, comprising at least one quaternary ammonium salt of imidazoline selected from the group consisting of those of formula (VI) below: 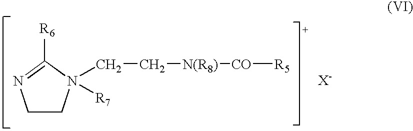
in which R₅ represents an alkenyl or alkyl radical containing from 8 to 30 carbon atoms, R₆ represents a hydrogen atom, a C₁-C₄ alkyl radical or an alkenyl or alkyl radical containing from 8 to 30 carbon atoms, R₇ represents a C₁-C₄ alkyl radical, R₈ represents a hydrogen atom or a C₁-C₄ alkyl radical, and X is an anion selected from the group consisting of the group of halides, phosphates, acetates, lactates, alkyl sulfates, alkyl sulfonates or alkylaryl sulfonates, R₇ denotes methyl and R₈ denotes hydrogen.

11. The composition as claimed in claim 1, wherein the cationic surfactant is selected from the group consisting of behenyltrimethylammonium chloride, cetyltrimethylammonium chloride, Quaternium-83, behenylamidopropyl-2,3-dihydroxypropyidimethylammonium chloride and palmitylamido-propyltrimethylammonium chloride.

12. The composition as claimed in claim 1, wherein the cationic surfactant is present in an amount of from 0.05% to 10% by weight relative to the total weight of the composition.

13. The composition as claimed in claim 1, wherein the cationic polymers are selected from the group consisting of those containing units comprising primary, secondary, tertiary and/or quaternary amine groups that may either form part of the main polymer chain or may be borne by a side substituent directly attached thereto.

14. The composition as claimed in claim 1, wherein said cationic polymers are selected from the group consisting of:
(1) homopolymers or copolymers derived from acrylic or methacrylic esters or amides and comprising at least one of the units of the following formulae: 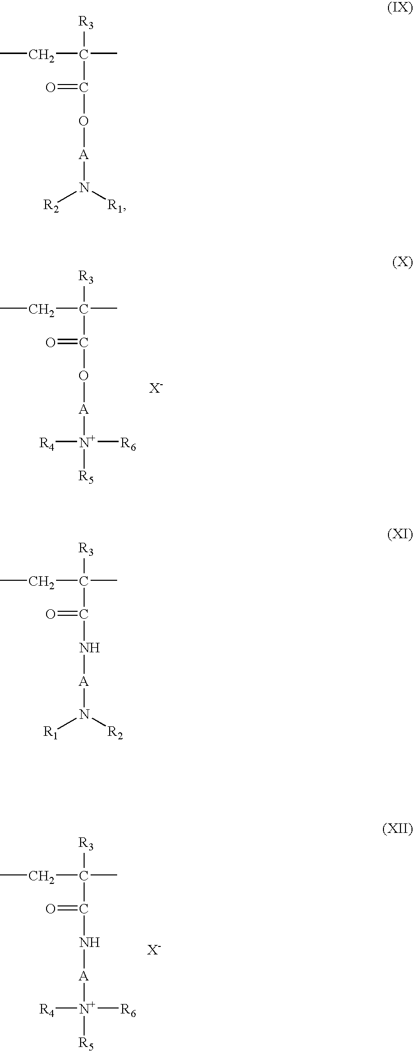
in which:
R₃, which may be identical or different, denote a hydrogen atom or a CH₃ radical;
A, which may be identical or different, represent a linear or branched alkyl group of 1 to 6 carbon atoms or a hydroxyalkyl group of 1 to 4 carbon atoms;
R₄, R₅ and R₆, which may be identical or different, represent an alkyl group containing from 1 to 18 carbon atoms or a benzyl radical;
R₁ and R₂, which may be identical or different, represent hydrogen or an alkyl group containing from 1 to 6 carbon atoms;
X denotes an anion derived from a mineral or organic acid,
(2) cationic polysaccharides,
(3) polymers consisting of piperazinyl units and of divalent alkylene or hydroxyalkylene radicals containing straight or branched chains, optionally interrupted by oxygen, sulfur or nitrogen atoms or by aromatic or heterocyclic rings, as well as the oxidation and/or quaternization products of these polymers,
(4) water-soluble polyaminoamides prepared in particular by polycondensation of an acidic compound with a polyamine; these polyaminoamides can be crosslinked with an epihalohydrin, a diepoxide, a dianhydride, an unsaturated dianhydride, a bisunsaturated derivative, a bishalohydrin, a bisazetidinium, a bishaloacyldiamine, a bisalkyl halide or alternatively with an oligomer resulting from the reaction of a difunctional compound which is reactive with a bishalohydrin, a bisazetidinium, a bishaloacyldiamine, a bisalkyl halide, an epilhalohydrin, a diepoxide or a bisunsaturated derivative; the crosslinking agent being used in proportions ranging from 0.025 to 0.35 mol per amine group of the polyaminoamide; these polyaminoamides can be alkylated or, if they contain one or more tertiary amine functions, they can be quaternized,
(5) polyaminoamides resulting from the condensation of polyalkylene polyamines with polycarboxylic acids followed by alkylation with difunctional agents,
(6) polymers obtained by reaction of a polyalkylene polyamine containing two primary amine groups and at least one secondary amine group with a dicarboxylic acid selected from the group consisting of diglycolic acid and saturated aliphatic dicarboxylic acids having from 3 to 8 carbon atoms,
(7) cyclopolymers of alkyldiallylamine or of dialkyldiallylammonium,
(8) quaternary diammonium polymers containing repeating units corresponding to the formula: 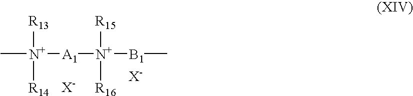
in which formula (XIV):
R₁₃, R₁₄, R₁₅ and R₁₆, which may be identical or different, represent aliphatic, alicyclic or arylaliphatic radicals containing from 1 to 20 carbon atoms or lower hydroxyalkylaliphatic radicals, or alternatively R₁₃, R₁₄, R₁₅ and R₁₆, together or separately, constitute, with the nitrogen atoms to which they are attached, heterocycles optionally containing a second hetero atom other than nitrogen, or alternatively R₁₃, R₁₄, R₁₅ and R₁₆ represent a linear or branched C₁-C₆ alkyl radical substituted with a nitrile, ester, acyl or amide group or a group —CO—O—R₁₇—D or —CO—NH—R₁₇—D where R₁₇ is an alkylene and D is a quaternary ammonium group;
A₁ and B₁ represent polymethylene groups containing from 2 to 20 carbon atoms, which groups may be linear or branched, saturated or unsaturated, and which may contain, linked to or intercalated in the main chain, one or more aromatic rings or one or more oxygen or sulfur atoms or sulfoxide, sulfone, disulfide, amino, alkylamino, hydroxyl, quaternary ammonium, ureido, amide or ester groups, and
X⁻ denotes an anion derived from a mineral or organic acid;
A₁, R₁₃ and R₁₅ can form, with the two nitrogen atoms to which they are attached, a piperazine ring; in addition, if A₁ denotes a linear or branched, saturated or unsaturated alkylene or hydroxyalkylene radical, B₁ can also denote a group (CH₂)ₙ—CO—D—OC—(CH₂)ₙ—in which D denotes:
a) a glycol residue of formula: —O-Z-O—, where Z denotes a linear or branched hydrocarbon-based radical or a group corresponding to one of the following formulae:
—(CH₂—CH₂—O)ₓ—CH₂—CH₂—
—[CH₂—CH(CH₃)—O]_{y}—CH₂—CH(CH₃)—
where x and y denote an integer from 1 to 4, representing a defined and unique degree of polymerization or any number from 1 to 4 representing an average degree of polymerization;
b) a bissecondary diamine residue such as a piperazine derivative;
c) a bisprimary diamine residue of formula: —NH—Y—NH—, where Y denotes a linear or branched hydrocarbon-based radical, or alternatively the divalent radical —CH₂—CH₂—S—S—CH₂—CH₂—;
d) a ureylene group of formula: —NH—CO—NH—;
(9) polyquaternary ammonium polymers consisting of units of formula (XVI): 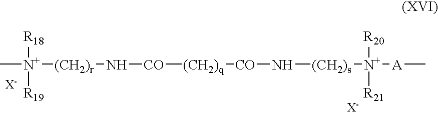
in which formula:
R₁₈, R₁₉, R₂₀ and R₂₁, which may be identical or different, represent a hydrogen atom or a methyl, ethyl, propyl, β-hydroxyethyl, β-hydroxypropyl or —CH₂CH₂(OCH₂CH₂)ₚOH radical,
where p is equal to 0 or to an integer between 1 and 6, with the proviso that R₁₈, R₁₉, R₂₀ and R₂₁ do not simultaneously represent a hydrogen atom,
r and s, which may be identical or different, are integers between 1 and 6,
q is equal to O or to an integer between 1 and 34,
X denotes a halogen atom,
A denotes a dihalide radical or represents —CH₂—CH₂—O—CH₂—CH₂—,
(10) quaternary polymers of vinylpyrrolidone and of vinylimidazole,
(11) polyamines,
(12) crosslinked methacryloyloxy(C₁-C₄)alkyltri(C₁-C₄)alkylammonium salt polymers, and
(13) polyalkyleneimines, polymers containing vinylpyridine or vinylpyridinium units, condensates of polyamines and of epichlorohydrin, polyquaternary ureylenes and chitin derivatives.

15. The composition as claimed in claim 14, wherein said cationic polymers are selected from the group consisting of cationic cyclopolymers, cationic polysaccharides and quaternary polymers of vinylpyrrolidone and of vinylimidazole, and mixtures thereof.

16. The composition as claimed in claim 15, wherein said cyclopolymer is selected from the group consisting of diallyidimethylammonium chloride homopolymers and copolymers of diallyidimethylammonium chloride and of acrylamide.

17. The composition as claimed in claim 16, wherein said cationic polysaccharides are selected from the group consisting of starches modified with a 2,3-epoxypropyltrimethylammonium salt, guar gums modified with a 2,3-epoxypropyltrimethylammonium salt and hydroxyethylcelluloses that have reacted with an epoxide substituted with a trimethylammonium group.

18. The composition as claimed in claim 17, wherein said quaternary polymers of vinylpyrrolidone and of vinylimidazole are selected from the group consisting of copolymers of vinylpyrrolidone and of methylvinylimidazolium salts.

19. The composition as claimed in claim 1, wherein the composition comprises at least one cationic polysaccharide and at least one quaternary polymer of vinylpyrrolidone and of vinylimidazole.

20. The composition as claimed in claim 1, wherein the composition comprises at least one diallyldimethylammonium chloride homopolymer and at least one quaternary polymer of vinylpyrrolidone and of vinylimidazole.

21. The composition as claimed in claim 1, wherein each cationic polymer is present in a concentration ranging from 0.001% to 20% by weight relative to the total weight of the composition.

22. The composition as claimed in claim 1, wherein said thickening polymer is selected from the group consisting of:
nonionic homopolymers and copolymers containing ethylenically unsaturated monomers of ester and/or amide type;
vinylpyrrolidone homopolymers or copolymers; and
polysaccharides.

23. The composition as claimed in claim 22, comprising a thickening polymer selected from the group consisting of polyacrylamides, methyl methacrylate/ethylene glycol dimethacrylate copolymers, butyl methacrylate/ methyl methacrylate copolymers and polymethyl methacrylates.

24. The composition as claimed in claim 22, comprising a vinylpyrrolidone homopolymer.

25. The composition as claimed in claim 22, comprising a polysaccharide selected from the group consisting of glucans, modified or unmodified starches, amylose, amylopectin, glycogen, dextrans, celluloses and derivatives thereof mannans, xylans, lignins, arabans, galactans, galacturonans, chitin, chitosans, glucuronoxylans, arabinoxylans, xyloglucans, glucomannans, pectic acids and pectins, arabinogalactans, carrageenans, agars, gum arabics, gum tragacanths, ghatti gums, karaya gums, carob gums, galactomannans and nonionic derivatives thereof, and mixtures thereof.

26. The composition as claimed in claim 1, wherein the thickening polymer is present in an amount of between 0.001% and 20% by weight relative to the total weight of the composition.

27. The composition as claimed in claim 1, further comprising at least one additional conditioner.

28. The composition as claimed in claim 27, wherein the additional conditioner is selected from the group consisting of silicones, carboxylic esters containing at least 12 carbon atoms, plant oils, mineral oils and synthetic oils, and mixtures thereof.

29. The composition as claimed in claim 1, comprising a cosmetically acceptable aqueous medium comprising said water and, optionally, a cosmetically acceptable solvent.

30. The composition as claimed in claim 29, wherein the cosmetically acceptable medium comprises asolvent selected from the group consisting of C₁-C₄ lower alcohols, alkylene glycols, polyol ethers, C₅-C₁₀ alkanes, acetone, methyl ethyl ketone, C₁-C₄ alkyl acetates, dimethoxyethane and diethoxyethane, and mixtures thereof.

31. The composition as claimed in claim 1, further comprising an additive selected from the group consisting of anionic, nonionic or amphoteric polymers, nonpolymeric thickeners, opacifiers, nacreous agents, vitamins, provitamins, waxes, natural or synthetic ceramides, fragrances, colorants, organic or mineral particles, preserving agents and pH stabilizers.

32. The composition as claimed in claim 1, which is in the form of a shampoo, a conditioner, a permanent-waving, relaxing, dyeing or bleaching composition for the hair or a rinse-out composition to be applied between the two steps of a permanent-waving or relaxing operation.

33. The composition according to claim 1, which is in the form of a rinse-out conditioner.

34. A method for conditioning or caring for keratin materials comprising applying thereto the composition of claim 1.

35. A method for giving hair sheen comprising applying to hair in need thereof the composition of claim 1.

36. A method for giving the hair suppleness, comprising applying to hair in need thereof the composition of claim 1.
